(19) United States
(12) Patent Application Publication
(10) Pub. No.: US 2019/0341141 A1
(43) Pub. Date: Nov. 7, 2019

(54) SEARCH AND VALUE ANALYSIS TOOL FOR HEALTHCARE PROVIDERS, PATIENTS, AND PAYERS

(71) Applicant: Mentis Healthcare Partners, LLC, Greenwood Village, CO (US)

(72) Inventors: William E. Younkes, Greenwood Village, CO (US); Suellyn M. Younkes, Greenwood Village, CO (US)

(21) Appl. No.: 16/400,576

(22) Filed: May 1, 2019

Related U.S. Application Data
(60) Provisional application No. 62/665,188, filed on May 1, 2018.

(51) Int. Cl. G16H 40/20; G16H 10/60; G06Q 40/08; G06Q 10/06; G06F 3/048

(52) U.S. Cl. CPC G16H 40/20; G16H 10/60; G16H 70/00; G06Q 10/06; G06F 3/048; G06Q 40/08

(57) ABSTRACT

An integrated search and value analysis tool for healthcare providers includes a scoring and reporting server in communication with a plurality of HIPAA compliant databases. A management engine running on the server may execute a database management module, a rule module, and a GUI module configured to display a GUI having a plurality of interactive, preconfigured screens at a healthcare service provider terminal, a patient terminal, a payer terminal, and an administrator terminal. In operation, the management engine may retrieve healthcare quality and cost data from a healthcare service provider system, store the quality and the cost data in the databases, value score the provider in relation to its peers, and report, via the preconfigured screens of the GUI, one or more value scores for the healthcare service provider, thereby enabling a provider selection that results in an optimized procedure outcome at an effective cost. Other embodiments are disclosed.

### REFERENCE TO PENDING PRIOR PATENT APPLICATION

This application claims the benefit under 35 U.S.C. 119 (e) of U.S. Provisional Patent Application No. 62/665,188, filed May 1, 2018 by William E. Younkes and Suellyn M. Younkes for “QUALITY AND COST ANALYSIS TOOL FOR HEALTHCARE PROVIDERS, PATIENTS, AND PAYERS,” all of which patent application is hereby incorporated herein by reference.

### BACKGROUND

Currently, the variance in outcome quality and cost for medical procedures in the United States can range from 100% to over 300%, depending on the healthcare service provider. A typical patient is generally unaware of these quality and cost, or value, variances. Today the patient does not have an effective way to protect themselves from receiving substandard medical care, and the health insurance provider or other payer (“payer”) is unprotected from unreasonably high medical costs.

Today a patient requiring medical treatment who wants to find the best healthcare service provider must consult a variety of discrete computerized technological platforms, such as, for example, online review services, medical concierge services/programs, and/or information websites and/or applications implemented by medical providers, provider groups, and/or insurer or payer networks. Both patients and payers endure a time-consuming research process that generally does not reveal or provide a technical assessment on quantitative quality or cost comparisons.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

One embodiment provides an integrated healthcare management tool for healthcare service providers, patients, and payers. The tool may include (1) a scoring and reporting server operating one or more scoring and reporting processors; (2) a database storing patient data, provider data, quality and cost data, and payer data; and (3) a management engine running on the scoring and reporting server, the management engine executing a database management module, a rule module including one or more scoring analysis algorithms, and a graphical user interface (GUI) module configured to display a GUI having a plurality of preconfigured, interactive screens to users at a provider terminal, a patient terminal, and a payer terminal, the management engine in communication with a healthcare service provider system of a healthcare service provider, the management engine: (a) receiving, from the healthcare service provider system, a set of quality and cost data associated with the healthcare service provider; (b) storing, in the database, the set of the quality and cost data; and (c) determining, based at least in part on the set of the quality and cost data, a value score for the healthcare service provider relative to a plurality of peer healthcare service providers.

Another embodiment provides a search and value analysis tool for healthcare providers, payers, and patients. The tool may include (1) a scoring and reporting server including at least one scoring and reporting processor communicating over a network with a patient terminal, a healthcare service provider system, a provider terminal, a payer terminal, and a plurality of HIPAA compliant databases storing participant patient data, healthcare provider data, healthcare quality and cost data, payer data, and healthcare value data; (2) a value scoring module operating on the at least one scoring and reporting processor, the value scoring module executing one or more scoring analysis algorithms for determining a value score for a healthcare provider based on the healthcare provider data and the healthcare quality and cost data as compared to a plurality of predefined quality and cost measures; and (3) a graphical user interface (GUI) module operating on the at least one scoring and reporting processor, the GUI module displaying the value score for the healthcare provider upon a plurality of preconfigured screens presented at the patient terminal, the payer terminal, and the healthcare provider terminal.

Yet another embodiment provides a method of technically evaluating a healthcare provider as compared to a plurality of peer healthcare providers using a search and value analysis tool having a scoring and reporting processor in communication with a database storing healthcare provider data and quality and cost data, the scoring and reporting processor including a management engine executing a value scoring module including one or more scoring analysis algorithms and a graphical user interface module (GUI) module configured to display a GUI having a plurality of preconfigured screens at a provider terminal and at a patient terminal. The method may include the following steps: (1) receiving, at the scoring and reporting processor from a healthcare service provider system, a set of quality and cost data relating to the healthcare provider; (2) determining, via the value scoring module, a value score for the healthcare provider based on the set of the quality and cost data as compared to a plurality of predefined quality and cost measures; and (3) displaying, via the plurality of the preconfigured screens of the GUI module, the value score at one of the provider terminal and the patient terminal.

Other embodiments are also disclosed.

Additional objects, advantages and novel features of the technology will be set forth in part in the description which follows, and in part will become more apparent to those skilled in the art upon examination of the following, or may be learned from practice of the technology.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIG. 1 provides a block diagram of an illustrative environment where various healthcare search and value analysis systems and methods may be implemented according to some embodiments;

FIG. 2 provides a schematic detailing a number of exemplary HIPAA compliant databases maintained at a healthcare services scoring and reporting center of the search and value analysis systems described herein;

FIG. 3 provides a block diagram depicting illustrative healthcare quality and cost collection, analysis, and reporting software according to some embodiments;

FIGS. 4A-4J provide a representative sampling of exemplary interactive and preconfigured screens of a graphical user interface (GUI) of the healthcare quality and cost collection, analysis, and reporting software of FIG. 3;

FIG. 5 provides a flowchart depicting an exemplary method of healthcare service provider value analysis and reporting according to some embodiments;

FIG. 6 provides a flowchart depicting a symptom search method in which a participant patient may search for healthcare providers that treat particular symptoms using embodiments of the search and value analysis system disclosed herein;

FIG. 7 provides a flowchart depicting an exemplary method of searching for a healthcare provider according to one or more conditions to be treated using embodiments of the search and value analysis system disclosed herein;

FIG. 8 provides a flowchart depicting an exemplary method of searching for a physician or a hospital that practices one or more medical specialties using embodiments of the search and value analysis system disclosed herein; and

FIG. 9 provides a flowchart depicting an exemplary method of searching for a hospital or urgent care facility having the nearest geo location relative to a participant terminal or a user-defined geo location using embodiments of the search and value analysis system disclosed herein.

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

Various embodiments of the systems and methods described herein generally relate to scoring healthcare service providers against their peers, thereby providing both patients and payers with an integrated tool for rapidly identifying the healthcare service providers providing the highest value to meet their needs in terms of both outcome quality and cost and providing healthcare service providers with valuable feedback that may be used to improve future outcomes and/or reduce costs. That is, embodiments of the disclosed systems and methods provide providers, patients, and payers with a searchable, integrated value analysis tool to identify, using a variety of search criteria, the healthcare service providers that have the highest probability for achieving the highest quality medical procedure outcomes at the most effective costs.

As discussed above in the Background section, existing technological solutions addressing the problem of computerized healthcare reviews and quality, cost, and/or value scorings generally consist of silo-type computerized platforms that review or compare healthcare service providers. Such silo-type computerized platforms include, for example, provider websites, payer/insurer websites or Intranet portals that provide provider summaries, ratings and/or rankings, and commercial Internet review sites that are open for public review, rating or scoring, ranking, and/or comment relating to healthcare service providers. Such silo-type platforms must be individually queried, with none of them providing any type of searchable results aggregation across a defined portion or an entirety of a healthcare services field. Further, existing silo-type platforms generally provide anecdotal reviews, rankings, and/or ratings/scores that represent discrete personal or institutional experiences, rather than quantitative, statistical quality and/or cost information and comparisons across a defined portion or an entirety of a healthcare services field. These isolated, silo-type platforms are also impossible to search in a comprehensive way that accommodates multiple criteria from a variety of sources, thereby acting as a barrier to quickly identifying the healthcare services provider that is right for the patient.

To provide a technological solution to the challenges presented by silo-type reviewing, rating, and/or ranking/scoring systems for healthcare service providers, the present systems and methods provide an integrated search and quality and cost—or value—analysis tool for healthcare providers, participant patients, and payers, where the payer may be a patient, an employer, or a commercial or government health insurance provider. The integrated tool aggregates quality and cost data, or value scoring information, provided by patients, healthcare service providers, and other third-party sources such as, for example, Healthgrades awards, analyzes value scoring information, and then provides a value score that compares each healthcare service provider against its peers, providing a tool to the participant patient and/or the payer to consult a single technological platform to rapidly identify the best healthcare service provider(s) for the participant patient's needs in terms of balancing the highest quality medical procedure outcomes at the most effective cost. The system also provides powerful search tools that allow the patient participant to navigate his or her medical needs, including searching for providers such as physicians and other facilities by name, location, and/or by specific symptoms, medical procedures provided, conditions treated, or medical specialties. Further, the system provides robust information tracking that enables comprehensive management of the patient participant's medical landscape, including tracking and linking the patient participant to his or her existing healthcare providers (e.g., treating physicians and pharmacy on record), healthcare insurance or other care plans, prescribed medications, and family members and emergency contacts.

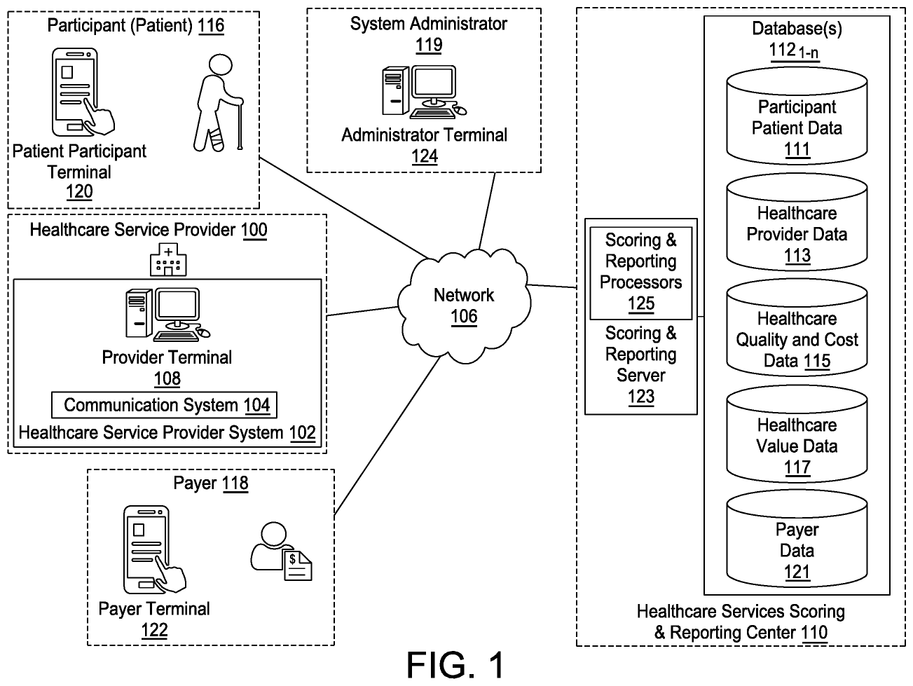

FIG. 1 provides a schematic depicting an illustrative environment where various techniques described herein may be implemented according to some embodiments. As shown, a healthcare service provider 100 such as, for example, a hospital, physician's office or clinic, urgent care facility, outpatient facility, or pharmacy. The healthcare service provider 100 may operate a healthcare service provider system 102 to obtain and store certain value scoring information including quality data and/or cost data collected over the course of patient treatment, including before, throughout, and/or after the provision of healthcare services to or the performance of a healthcare procedure upon a patient. Exemplary quality data may include, by way of limited example, patient statistics and corresponding recorded procedure outcomes, mortality or morbidity rates, referral rates, etc. Exemplary cost data may include, for example, a cost or costs associated with the procedures performed by that healthcare service provider.

In some embodiments, the healthcare service provider system 102 may incorporate a communication system 104 that provides communication abilities to the healthcare service provider 102. To that end, the communication system may include one or more analog switches, servers, IP gateways, PBX systems, etc. For example, in some embodiments, the communication system 104 may be operable to provide communications through a network 106, which may include, for example, the Internet. Additionally or alternatively, the network may include wireless cellular networks or the like.

To enable the healthcare service provider system 102 to communicate via the communication system 104 and the network 106, the service provider 100 may operate a network-enabled provider terminal 108 configured to securely compile and transmit the quality and cost information, or value scoring information, along with healthcare provider data such as the provider's location, hours of operation, procedures offered, conditions treated, medical specialties practiced, insurance providers and/or plans accepted, and so on to and from other system components. The provider terminal 108 may be any appropriate network-enable user device such as, for example, a desktop computer, laptop computer, tablet computer, smartphone, or the like.

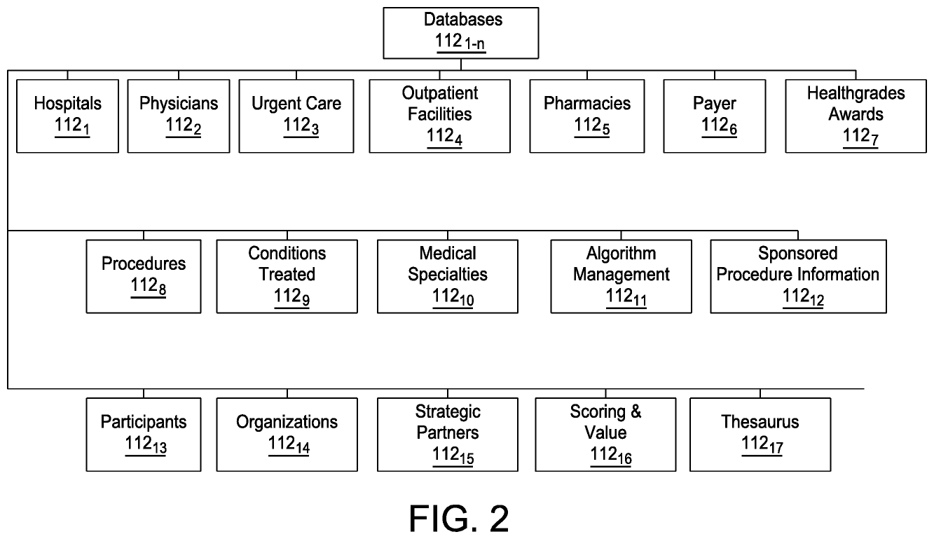

In one embodiment, a healthcare services scoring and reporting center 110, a payer 118, and a system administrator 119 may be communicatively coupled with the healthcare service provider 100 via the network 106. In some embodiments, the healthcare services scoring and reporting center 110 may include a plurality of HIPAA compliant databases 112₁₋ₙ (FIG. 2) storing participant patient data 111, healthcare provider data 113, healthcare quality and cost data 115, payer data 121, and healthcare value data 117. FIG. 2 provides a schematic detailing a number of exemplary HIPAA compliant databases 112₁₋ₙ maintained at the healthcare services scoring and reporting center 110 including a hospitals data database 112₁, a physicians database 112₂, an urgent care database 112₃, an outpatient facilities database 112₄, a pharmacies database 112₅, a payer database 112₆, a Healthgrades awards database 112₇, a procedures database 112₈, a conditions treated database 112₉, a medical specialties database 112₁₀, an algorithm management database 112₁₁, a sponsored procedure database 112₁₂, a participants database 112₁₃, an organizations database 112₁₄, a strategic partners database 112₁₅, a scoring and value database 112₁₆, and a synonym or thesaurus database 112₁₇. Each of the databases 112₁₋ₙ may be communicatively coupled or cross-referenced with one another and with a scoring and reporting server 123 running one or more and scoring and reporting processors 125, as shown in the schematic of FIG. 1.

Generally, the databases 112₁₋ₙ may include any suitable type of application or data structure that may be configured as a data repository. For example, the databases may be configured as relational databases that include one or more tables of columns and rows that may be searched or queried according to a query language, such as a version of Structured Query Language (SQL). Alternatively, the databases may be configured as structured data stores that include data records formatted according to a markup language, such as a version of eXtensible Markup Language (XML). In other embodiments, the databases may be implemented using arbitrarily or minimally structured data files managed and accessible through any suitable type of application.

Each of the databases 112₁₋ₙ may include any appropriate information provided by the healthcare service provider 100, the participant patient 116, the payer 118, and/or the system administrator 119. For example, in one embodiment, the hospitals database 112₁ may include for each entry a name, address, description, industry ratings, physician listings, insurance providers accepted, conditions treated, medical specialties, procedures provided, cost and quality data, and value scores computed for the hospital. The physicians, urgent care, outpatient facilities, and pharmacies databases 112₂₋₅ may include similar information for each of the respective healthcare service providers. In one embodiment, the payer database 112₆ may include for each entry a listing of insurance plans offered, their associated costs, the medical coverage provided under each of the plans offered, and the medical providers that accept each of the plans. The Healthgrades awards database 112₇ may include a number of industry recognized Healthgrades awards for assignment, if applicable, to select healthcare providers 100, including, by way of limited example, America's 100 Best Hospitals for Critical Care, America's 100 Best Hospitals for Gastrointestinal Care, America's 50 Best Hospitals, General Surgery Excellence, Patient Experience Award, and so on. The procedures database 112₈ may include a list of medical procedures by name, along with, for example, cost information, quality information, provider availability information, and recovery information for each. The conditions treated database 112₉ may including a list of treatable conditions such as, for example, abscess, acidosis, acoustic neuroma, abdominal pain, and so on. In one embodiment, the conditions treated database may include over 11,000 conditions along with cost information, quality information, provider availability information, and more for each. The medical specialties database 112₁₀ may include a listing of thousands of medical specialties practiced by physicians or other providers along with cost information, quality information, provider availability information, and more for each. Examples include abdominal radiology, academic medicine, acupressure, acupuncture, acute care medicine, and so on. The sponsored procedure information database 112₁₂ may include information regarding sponsored medical procedures such as clinical trials and their sponsors. The participants database 112₁₃ may include for each participant patient 116 a name, contact information, an associated payer (e.g., insurance provider), prescribed medications, treating doctors, associated pharmacies, family members, emergency contacts, medical conditions, and more. The organizations and strategic partners databases 112₁₄₋₁₅ may include a name, organization designation, location, website, and other information associated with all of the organizations partnered with the system, including advertisers, medical provider organizations, sponsors, advertisers, and more. The algorithm management database 112₁₁ may include scoring analysis algorithm(s), reporting algorithms, and a variety of search algorithms executed by the scoring and reporting processors 125, discussed further below. The scoring and value database 112₁₆ may include weighted quality and cost scores—or value scores—determined by the system from the value scoring information provided and/or collected for each of the procedures, medical specialties, and/or conditions treated, along with composite value scores for healthcare providers as a whole. The value score may include only quality scores, or may include a combination of weighted quality and cost scores. The thesaurus database 112₁₇ may include a variety of normalized medical terms used in the healthcare industry, each cross-referenced to one or more general or colloquial counterparts. For example, the search criteria “breast diagnostic test” may be linked to the normalized medical search criteria “mammogram” and vice versa. The colloquial term “breast cancer surgery” may be linked to the normalized medical term “mastectomy” and vice versa. Information contained within the thesaurus database 112₁₇ may be used by the scoring and reporting server 123 and processors 125 to provide more robust searching capabilities in which a user input of a general or colloquial term may be used to query normalized medical terms associated with symptoms, procedures, conditions treated, and/or medical specialties, as discussed further below.

As shown in FIG. 1, the participant (e.g., a patient) 116, the payer 118, and the system administrator 119 may also access respective participant, payer, and administrator terminals 120, 122, 124 that are configured to securely communicate with the scoring and reporting server 123 for the purpose of transmitting, receiving, and/or viewing/displaying data and otherwise communicating with the server 123. Like the provider terminal 108, the participant, player, and administrative terminals 120, 122, 124 may be any appropriate network-enabled electronic user devices such as, for example, desktop computers, laptop computers, tablet computers, smartphones, and so on.

During operation of some embodiments, the participant patient data 111, healthcare provider data 113, healthcare quality and cost data 115, and payer data 121 may be collected by an appropriate one of the participant 116, the provider 100, the payer 118, and/or the system administrator 119 and transmitted via the participant terminal 120, the provider terminal 108, the payer terminal 122, and/or the administrator terminal 124 to the healthcare services scoring and reporting center 110 and stored in the appropriate one of the cross-referenced databases 112₁₋ₙ, discussed above. The scoring and reporting server 123 and processors 125 may access the databases 112₁₋ₙ to perform complex search requests submitted by any of the participant 116, the provider 100, the payer 118, and/or the administrator 119 to execute one or more scoring analysis algorithms to value score the healthcare service provider 100 (e.g., a physician, a pharmacy, a hospital, etc.) based on the healthcare quality and cost data and a plurality of predefined quality and cost measures based on standard elements of care and the healthcare service provider's demonstrated ability to meet the standards, resulting in value scores associated with the healthcare service provider. The value scoring information and the value scores may be stored in the HIPAA compliant scoring and value database 112₁₆, from where it may be further accessed and analyzed and/or processed by the scoring and reporting server 123 and processors 125 for reporting to the participant 116 and/or to the payer 118 via a reporting module (e.g., a reporting application module) running on or accessible by the payer and/or the patient terminals, respectively.

The databases 112₁₋ₙ, as well as the scoring and reporting server 123 and processors 125 may be co-located at the healthcare services scoring and reporting center 110, as illustrated in FIG. 1. In some embodiments, these components, or sub-components thereof, may be combined or distributed in any appropriate manner across multiple locations and/or distributed computing platforms.

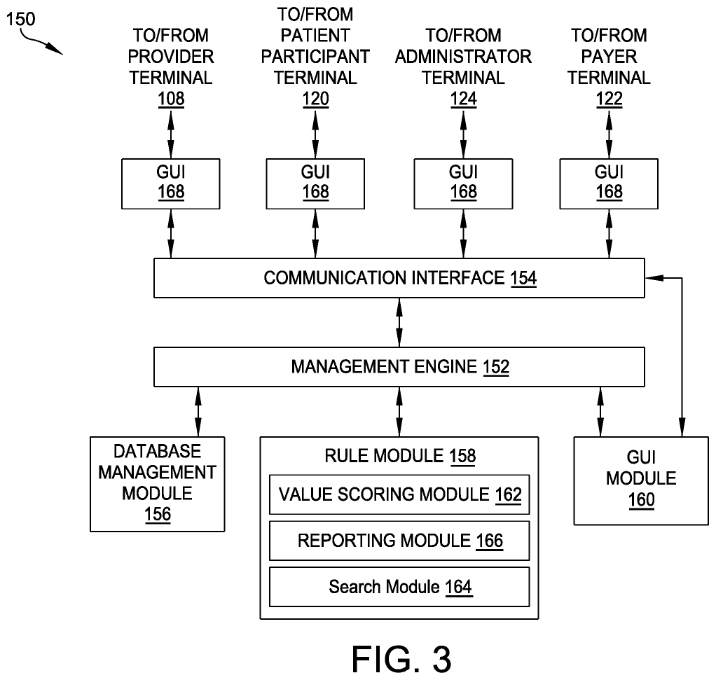

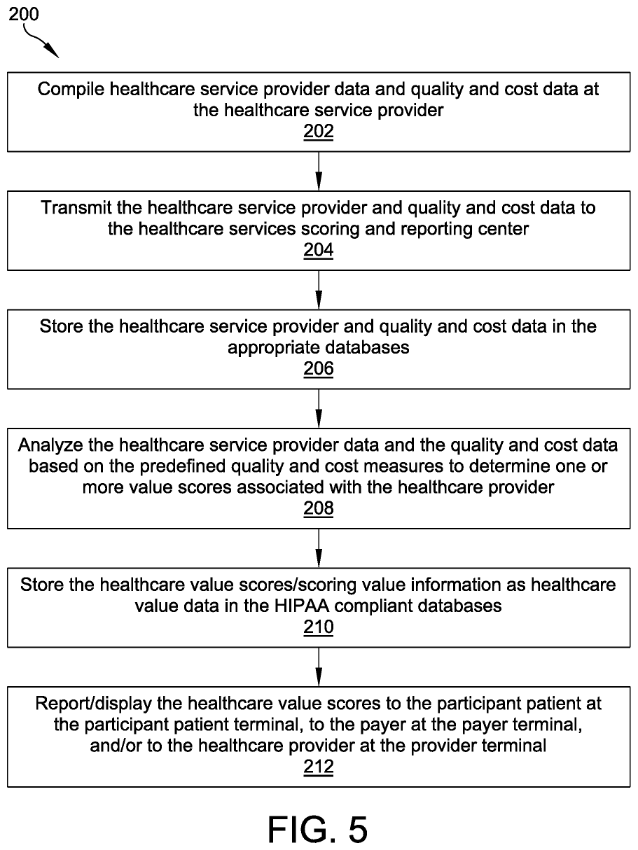

FIG. 3 provides a block diagram depicting illustrative healthcare quality and cost collection, analysis, and reporting software 150 according to some embodiments. In various implementations, embodiments of such software 150 may be executed by the processors 125 of the scoring and reporting server 123. As shown, a management engine 152 may be coupled to a communication interface 154, a database management module 156, a rule module 158, and a graphical user interface (GUI) module 160 operatively coupled with the provider terminal 108, the participant terminal 120, the payer terminal 122, and the administrator terminal 124 via the communication interface 154. The management engine 152 may be configured to perform a variety of operations including those described below with respect to FIGS. 5-9, discussed below.

The communication interface 154 may enable the quality and cost collection, analysis, and reporting software 150 to securely exchange information with other systems and/or system components, including the healthcare service provider system 102 and the provider terminal 108, the participant (e.g., patient) terminal 120, the administrator terminal 124, and/or the payer terminal 122 via the network 106 (FIG. 1). In some embodiments, the communication interface 154 may be configured to transmit and/or receive information using secure socket layer (SSL) encryption. Additionally or alternatively, other connections may also be used, such as, for example, XML file transmission utilizing file transfer protocol (FTP), hypertext transfer protocol (HTTPS) POST transactions, or other data transmission protocols. The communication interface may further include any of a variety of standardized application programming interfaces (APIs) configured to allow different software programs to communicate (e.g., to request services and respond to such requests) in an autonomous, web-based, and/or platform-independent manner. For example, the healthcare service provider system 102 may expose certain data (e.g., patient procedures offered, patient procedure outcomes, procedure costs, medical specialties, Healthgrades awards received, insurances accepted) via a HIPAA compliant, secured web interface. The communication interface 154 may then access the exposed data and/or functions via the appropriate API(s).

In various embodiments, the modules shown in FIG. 3 may represent sets of software routines, logic functions, and/or data structures that are configured to perform specified operations. Although these modules are shown as distinct logical blocks, in other embodiments, at least some of the functionality provided by these modules may be combined into fewer blocks or parceled into additional blocks. Conversely, any given one of the modules may be implemented such that its functionality is divided among two or more logical blocks. Moreover, although shown with a particular configuration, in other embodiments these various modules may be rearranged in other suitable ways.

The database management module 156 may include any suitable database management system (DBMS) or application configured to manage the creation, maintenance, and use of the participant patient data 111, the healthcare provider data 113, the healthcare quality & cost data 115, the payer data 121, and the healthcare value data 117 stored in the databases 112₁₋ₙ (FIGS. 1-2).

The rule module 158 may include one or more sets of rules, in any suitable format, that provide a framework for the analysis, searching, and reporting of the participant patient data 111, the healthcare provider data 113, the healthcare quality & cost data 115, the payer data 121, and/or the healthcare value data 117 that is collected from the healthcare service provider 100, the participant 116, the payer 118, and/or the system administrator 119, or that is determined by the server 123 and processors 125, as described in further detail below. For example, the rule module 158 may include a value scoring module 162 that incorporates one or more scoring analysis algorithms and the predefined quality and cost measures by which healthcare service providers 100 may be compared and value scored according to the one or more scoring analysis algorithms. The rule module may also include a search module 164 configured to execute a number of search algorithms that enable complex searching of the databases 112₁₋ₙ, allowing users to search for healthcare providers (e.g., doctors and/or facilities) according to various search criteria such as, for example, procedures provided, medical specialties, location, cost, value score, insurance plans/providers accepted, or even a combination of a number of relevant search criteria, as discussed further below. In searching, users may input a variety of colloquial or general search criteria, and the search module 164 may access the synonym or thesaurus database 112₁₇ to cross-reference the input terms to one or more normalized medical search criteria to achieve more effective search results within the procedures, conditions treated, and/or medical specialties databases 112₈₋₁₀. The rule module 158 may also include a reporting module 166 including instructions for reporting or displaying information, including the search results and provider value scores, to the participant patient terminal 120, the payer terminal 122, the healthcare provider terminal 108, and/or the administrator terminal 124.

The GUI module 160 may be configured to provide, for example, a web-based user interface (WUI) that implements JAVA®, AJAX®, ADOBE FLEX®, MICROSOFT.NET®, or similar technologies to provide real-time user control. The user interface may be implemented on mobile technology that includes various operating systems, which may include, but are not limited to, Apple's iOS operating system and Google's Android operating system. In other cases, the GUI module may implement a command line interface, a network-enabled application interface, or another suitable interface using non-web-based technologies.

In various embodiments, a GUI 168 may be displayed to users via the GUI module 160 of FIG. 2. The GUI 168 may be operable to display information and/or receive commands from the user at the participant patient terminal 120, the provider terminal 108, the payer terminal 122, and/or the administrator terminal 124. In various implementations, the GUI 168 may be displayed, via a number of appropriate preconfigured and interactive screens, to the healthcare service provider 100 at the provider terminal 108, to the participant patient 116 at the participant patient terminal 120, to the payer 118 at the payer terminal 122, and/or to the system administrator 119 at the administrator terminal 124 via the communication interface 154 and the network 106. For example, value scores associated with a healthcare provider 100 or its particular procedures, medical specialties, and/or conditions treated may be reported and displayed via the GUI module 160 upon one or more preconfigured screens at the payer terminal 122 and/or the participant patient terminal 120. The GUI module 160 may also display one or more preconfigured screens at the payer terminal 122 and/or the patient terminal 120 that enable querying of the scoring and reporting server 123 such that, for example, the participant patient 116 may query the scoring and reporting server 123 regarding specific physicians, hospitals, or other medical facilities, or for specific symptoms, medical procedures, medical specialties, and more, thereby enabling the participant patient 116 and/or the payer 118 to find the most applicable and highest quality healthcare provider at the most effective cost.

In another example, the GUI module 160 may display one or more preconfigured screens at the participant patient terminal 120 that enable the participant 116 to create and submit a survey to the healthcare service provider system 104 requesting specific data in performing the patient's elected procedure. In reviewing the survey response data, including cost, expected complications, mortality, length of stay, co-morbidities, etc., the participant patient 116 may be assisted in selecting a healthcare provider that performs the best overall.

In yet another example, the GUI module 160 may be configured to display one or more preconfigured screens at the payer terminal 122 that enable direct communication with the healthcare service provider 100 at the provider terminal 108 to enable a number of functions such as initiating a payment of an invoice from the payer 118 to the healthcare service provider 100 or initiating a request for the healthcare service provider 100 to transmit a patient's medical records to the healthcare services scoring and reporting center 110 for storage in one of the HIPAA compliant databases 112₁₋ₙ.

A browser or application window on any of the provider, patient, and/or payer terminals may be configured to display text content, image content, input features, navigable links, etc. of the preconfigured screens of the GUI. Each preconfigured screen may include any appropriate type of content in various combinations, and the screen(s) displayed to the users may be specific to the viewing platform. For example, the screen(s) presented at the provider terminal may differ from the screen(s) shown at the patient terminal, which may differ from the screen(s) shown at the payer terminal, depending on a variety of factors including, for example, the type of information to be collected or transmitted, security concerns, user permissions, and so on.

GUI screen content may be interspersed or combined in any suitable fashion according to the capabilities of the browser and language used to implement the GUI 168, and may be displayed in any suitable area of the browser or application window. In some embodiments, the window may be generated and managed by a web browser such as, for example, MICROSOFT EXPLORER®, FIREFOX®, SAFARI®, CHROME®, etc., implemented from the healthcare services ranking and reporting center.

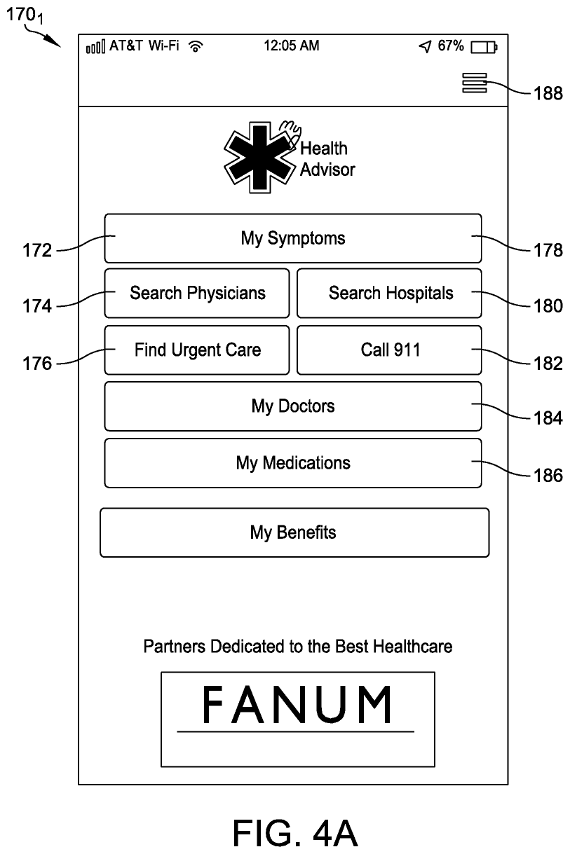

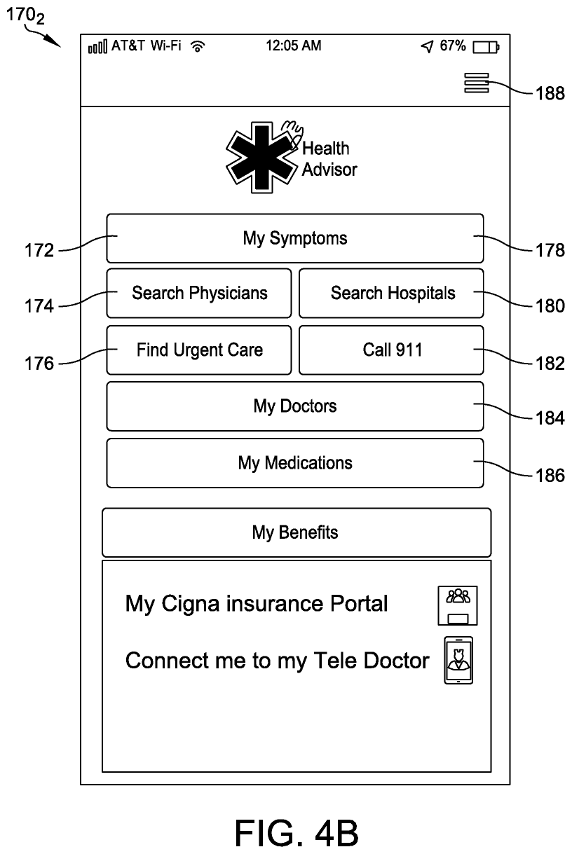

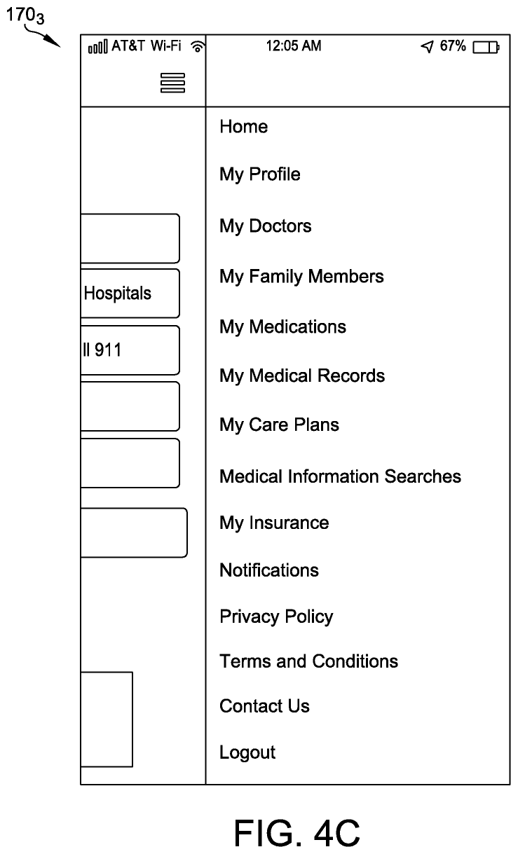

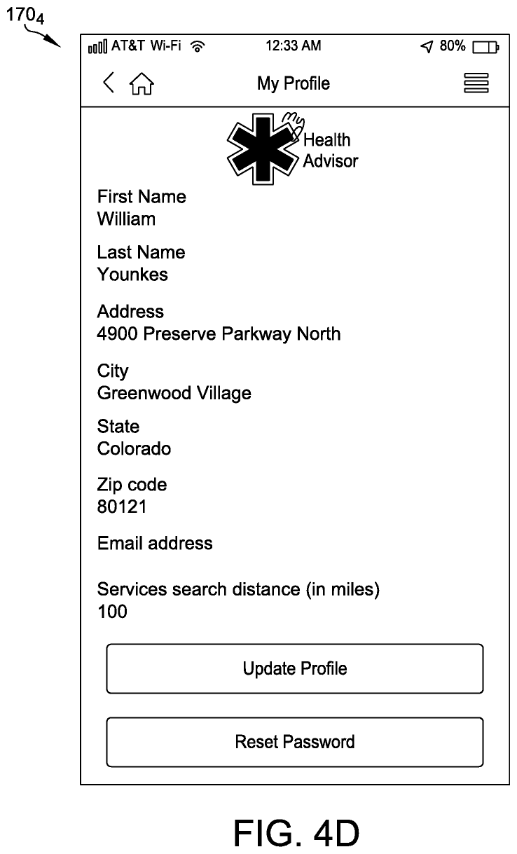

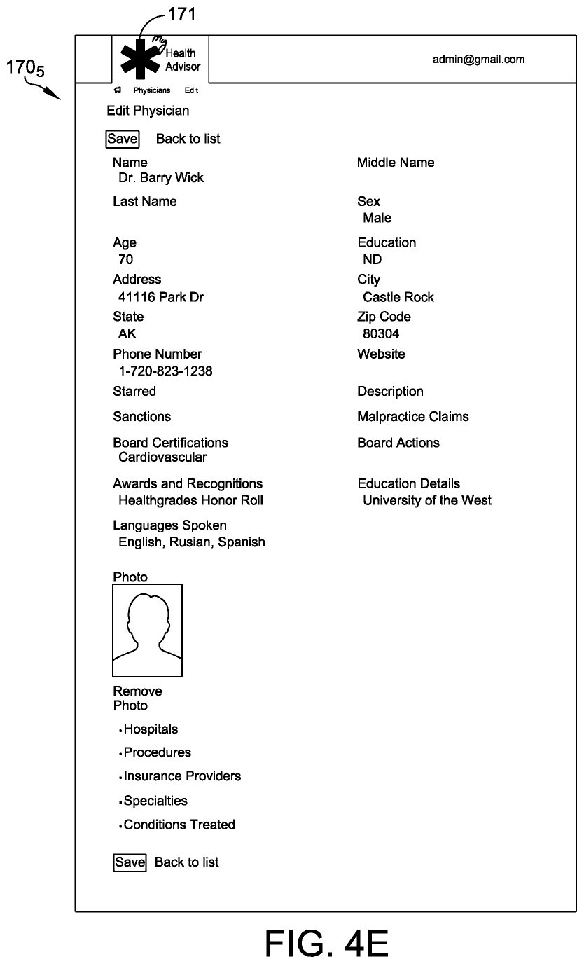

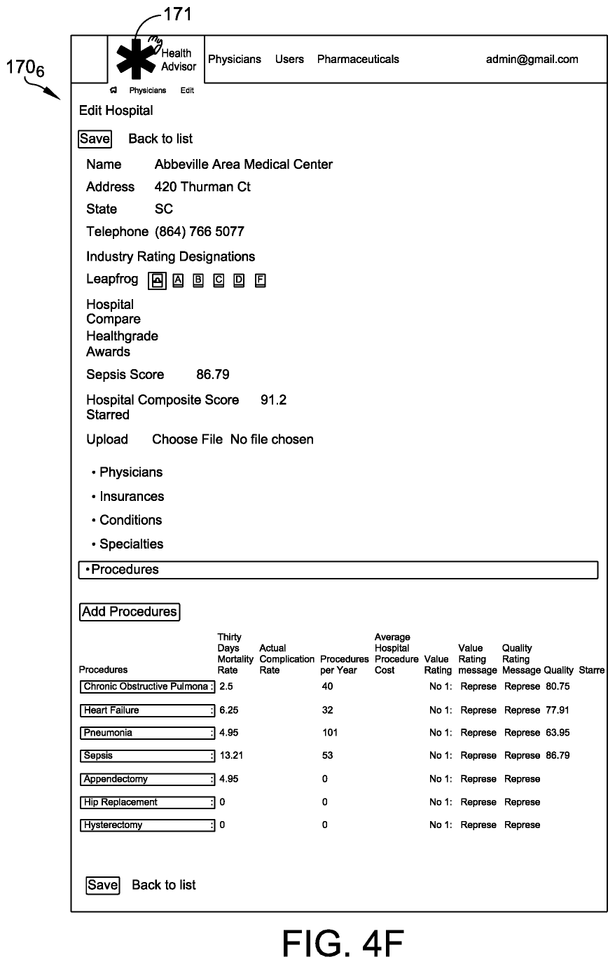

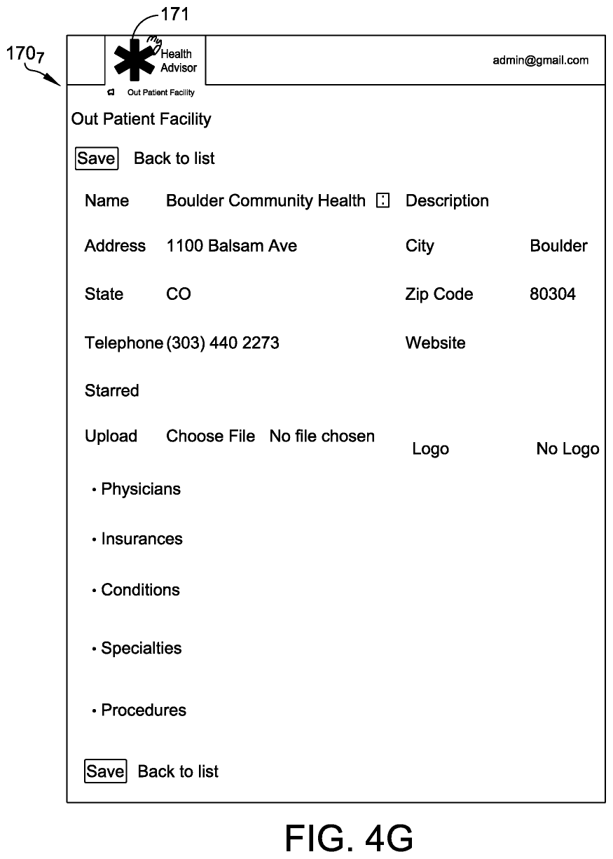

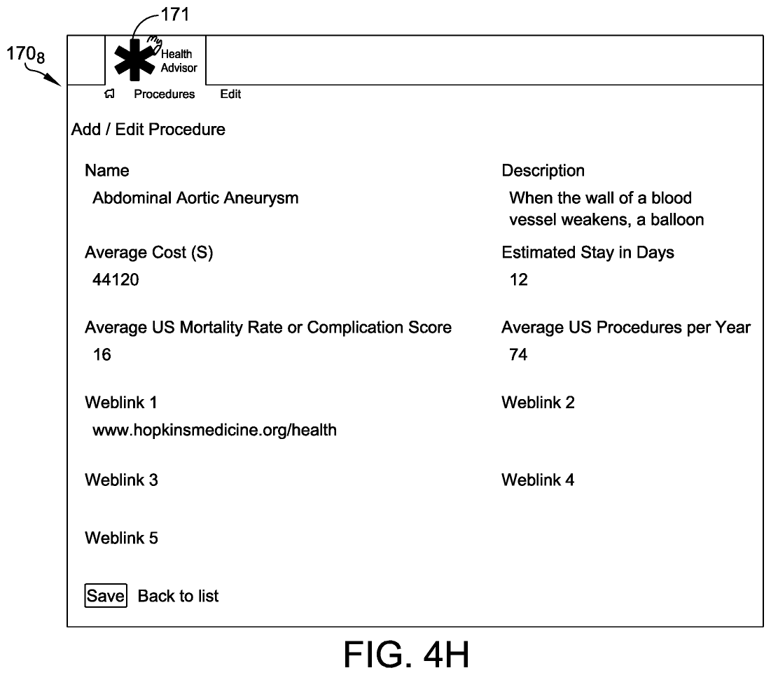

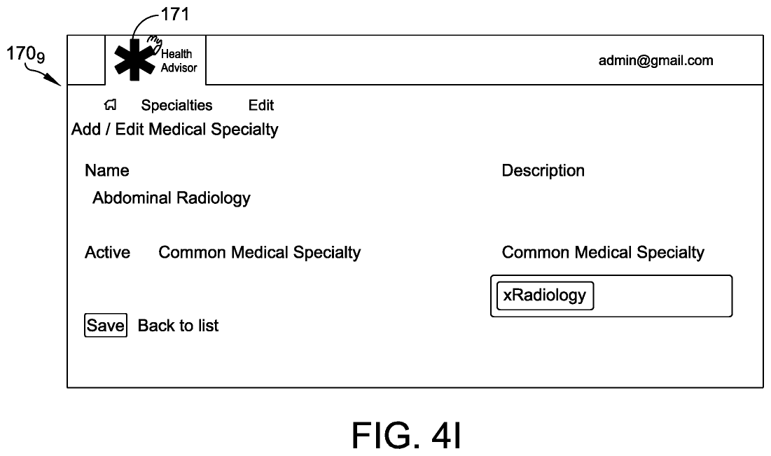

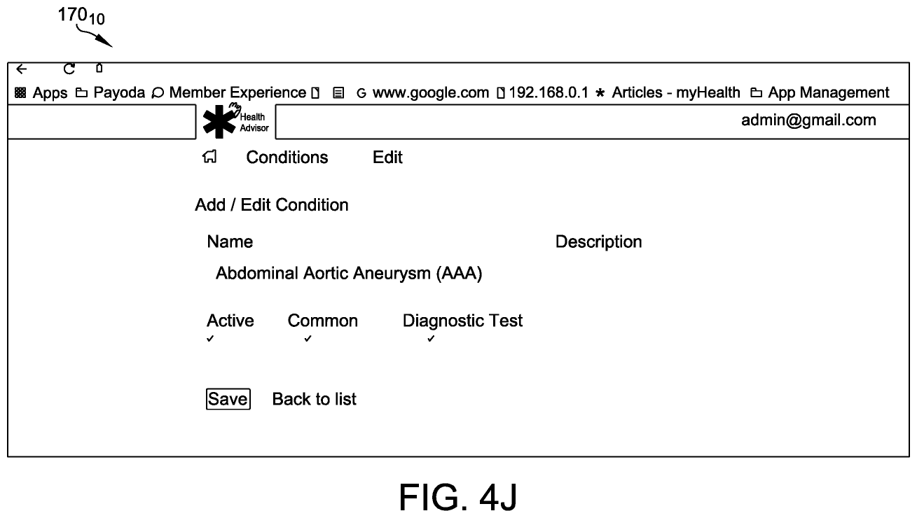

To demonstrate, FIGS. 4A-4J provide a representative sampling of exemplary interactive and preconfigured screens 170₁₋ₙ of the GUI 168 of the healthcare quality and cost collection, analysis, and reporting software 150. Specifically, FIG. 4A depicts an exemplary home screen 170₁, which includes links to several different search screens including: a “my symptoms” link 172, through which a user may search healthcare providers based on one or more symptoms; a “search physicians” link 174, through which the user may search individual physicians according to a number of criteria including, for example, name, location, medical specialty, conditions treated, procedures provided, etc.; a “find urgent care” link 176, through which the user may search urgent care facilities according a number of criteria including a nearest geo location as well as a name, location, medical specialty, conditions treated, procedures provided, etc.; a “search hospitals” link 178, through which the user may search hospitals according to a variety of criteria including a nearest geo location as well as a name, location, medical specialty, conditions treated, procedures provided, etc.; a “call 911” link 180, through which the user may directly contact emergency services; a “my doctors” 182 link including a listing and details of the participant's treating healthcare providers; a “my medications” link 184 including a listing of the participant's prescribed medications; and a “my benefits” link 186 linking to an insurance portal for the participant's insurance plan and connecting to an affiliated tele doctor program, as shown the exemplary expanded home screen 170₂ of FIG. 4B.

In this embodiment, the home screen 170₁ may also include a menu icon 188, which may be expanded as shown in the expanded home screen 170₃ of FIG. 4C to include links to a number of preconfigured record keeping, search, and notification screens.

In further examples, FIG. 4D depicts an exemplary “my profile” screen 170₄, through which the participant patient 116 may add his or her patient details, including a name, an address, and associated insurance plans, medications, treating doctors, family member profiles, and so on. FIGS. 4E-4G depict exemplary physician, hospital, and out-patient facility profile screens 170_{5,6,7}, respectively. Each of the profile screens 170_{5,6,7} allow the particular healthcare provider 100 or the administrator 119 to record any pertinent details relevant to the provider 100, including, for example, a name, location and contact information, industry rating designations, insurance providers accepted, Healthgrade awards, affiliated hospitals, medical facilities, or physicians, medical specialties, medical procedures provided, medical conditions treated, and value scoring information or cost and quality data related to the providers healthcare services. The profile screens may also include respective value scores for the provider 100, as computed by the value scoring module 162. The value scores may apply to the provider 100 as a whole or on a more granular level to specific procedures, medical specials, individual physicians, etc. In one embodiment, healthcare providers 100 who opt to share value scoring information with the system may be designated with a star icon 171 upon any preconfigured GUI screens featuring the provider, representing to the user that the provider 100 is a partner is providing quantitative value scoring information to the system.

FIGS. 4H-4J depict exemplary addition/edit screens 170_{8,9,10}, for medical procedures, medical specialties, and conditions treated, respectively. Using the addition/edit screens, users such as the healthcare provider 100, payer 118, and/or the system administrator 119 may add and/or edit individual medical procedures, medical specialties, and treated conditions that may be stored in the respective databases and linked to healthcare providers 100 that provide the procedures, specialize in the specialties, and/or treat the conditions.

The various systems shown in FIGS. 1-3 and 4A-4J enable the execution of methods relating to the collection, management, analysis, and reporting of healthcare service provider participant patient data 111, healthcare provider data 113, healthcare quality and cost data 115, payer data 121, and healthcare value data 117, the scoring of healthcare service providers 100 in relation to their peers, and the sophisticated searching and reporting of provider participant patient data 111, healthcare provider data 113, healthcare quality and cost data 115, payer data 121, and healthcare value data 117. In addition to enabling the participant patient's comprehensive management of his/her health, such reporting also identifies areas where healthcare spending can be reduced to enable patients to achieve the best possible outcomes for the lowest healthcare costs. Generally speaking, such methods may include information collection, information analysis and provider scoring, and reporting of scoring, patient healthcare spending, and value information to the payer 118, the participant patient 116, and/or the healthcare service provider 100.

FIG. 5 provides a flowchart depicting an exemplary method (200) of healthcare service provider quality and cost, or value, analysis and reporting according to some embodiments. In one embodiment, the method (200) may initiate with the healthcare service provider 100 utilizing the GUI 168 presented at the healthcare service provider terminal 108 to compile healthcare provider data 113 associated with the provider 100 (e.g., name, location, procedures provided, conditions treated, medical specialties, insurance providers accepted, etc.) and quality and cost data 115 from patient records (202) (e.g., procedure costs, procedure mortality rates, procedure recovery times, etc.) and transmit the data to the healthcare services scoring and reporting center 110 via the network 106 (204) for storage in the HIPAA compliant databases 112₁₋ₙ (206).

The healthcare service provider data 113 and the quality and cost data 115 may then be analyzed by the management engine 152 (e.g., running on the processors 125 of scoring and reporting server 123) implementing the plurality of scoring analysis algorithm(s) set forth in the value scoring module 162 of the rule module 158 (208), thereby determining one or more value scores associated with the healthcare service provider 100 based on specific and predefined quality and cost measures for procedures, treated conditions, medical specialties, and/or comprehensive standards promulgated for that category of provider. In one embodiment, the healthcare provider 100 may be associated with numerous value scores. For example, as shown in the hospital information screen shot 170₆ of FIG. 4F, a hospital may have an industry rating of A-F and several value scores weighted by cost and quality, including, for instance, a hospital composite score (91.2) and individual scores relating to procedures offered, conditions treated, or medical specialties such as a pneumonia score (63.95), a sepsis score (91.2), and a heart failure score (80.75).

The value score(s) determined by the management engine 152 (208) may then be stored as healthcare value data 117 in the HIPAA compliant scoring and value database 112₁₆ (210). The stored healthcare value data 117 may be further manipulated by the management engine 152 (e.g., running on the scoring and reporting server 123) implementing one or more reporting algorithm(s) defined in the reporting module 166 and/or the GUI module 160 to report or display the healthcare value data 117 in any appropriate manner via the preconfigured screens of the GUI 168 to the participant patient 116 at the participant patient terminal 120, to the payer 118 at the payer terminal 122, and/or to the healthcare provider 100 at the provider terminal 108 (212).

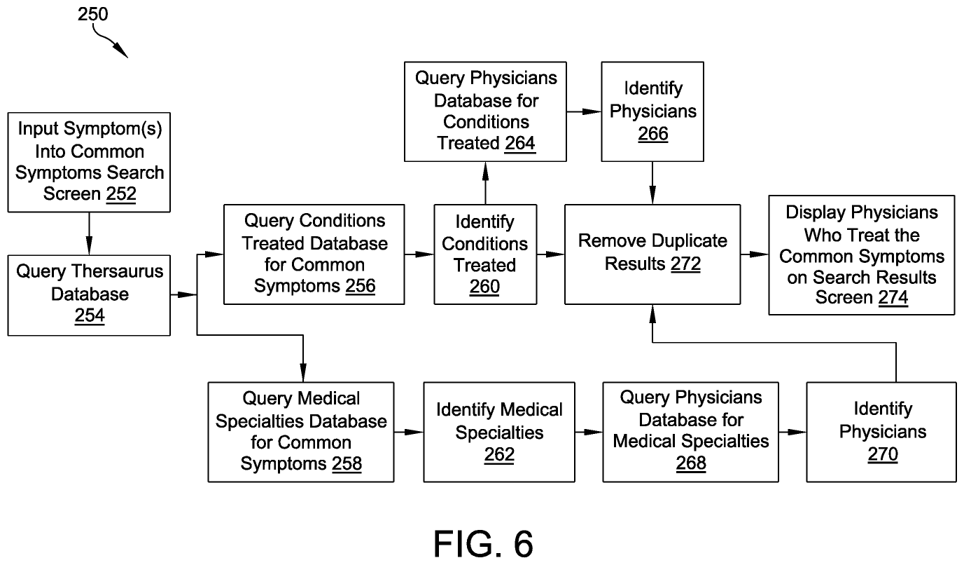

FIGS. 6-9 provide flowcharts depicting exemplary search methods carried out using embodiments of the healthcare search and value analysis tool described herein. Using embodiments of the search and value analysis tool disclosed herein, users may search for the highest value healthcare providers 100 according to a variety of search parameters, including, for example, one or more symptoms, procedures, medical specialties, conditions treated, and/or according to a geo location of the provider 100 relative to the participant 116.

More specifically, FIG. 6 provides a flowchart depicting a symptom search method (250) in which a participant patient 116 may search for healthcare providers that treat particular symptoms. In this embodiment, the method (250) may begin with the participant patient 116 inputting one or more common symptoms (e.g., allergies, asthma, back pain, blurry vision, heartburn, stomach cramps, etc.) into a preconfigured and interactive symptom search screen presented at the participant terminal 120 via the GUI 168 (252). The method (250) may continue with the search module 164 querying the thesaurus database 112₁₇ for the common symptoms search criteria input by the user to determine if one or more normalized medical search criteria correlate with or to the symptoms input by the user for use in performing the symptom search (254). For example, the participant patient 116 may input the common symptom “heartburn,” and the search module 164 may query the thesaurus database 112₁₇ to determine the additional and/or replacement normalized symptom “acid reflux” for subsequent use in searching other databases such as the conditions treated and/or medical specialties databases 112₉₋₁₀.

Once the thesaurus database 112₁₇ has been searched (254), the search module 164 may simultaneously query the conditions treated database 112₉ (256) and the medical specialties database 112₁₀ (258) for the common symptoms, including both the input and the normalized search terms, to identify a list of one or more conditions treated that are associated with the common symptoms (260) and a list of one or more medical specialties that are associated with the common symptoms (262). Next, the search module 164 may query the physicians database 112₂ for the identified conditions treated (264) to determine a list of one or more physicians that treat the identified conditions treated (266) and may separately query the physicians database 112₂ for the identified medical specialties (268) to determine a list of one or more physicians that specialize in the identified medical specialties (270). Duplicate results may be removed (272), resulting in a list of physicians who treat the common symptoms originally input into the search screen. The reporting module 166 may display the results to the participant 116 through a search results screen displayed via the GUI 168 at the participant terminal 120 (274). In one embodiment, the displayed results may include one or more value scores associated with each of the physicians appearing in the search results, and the results may be ordered according to a highest value such that the results prioritize a highest value provider, or the provider with the highest value score.

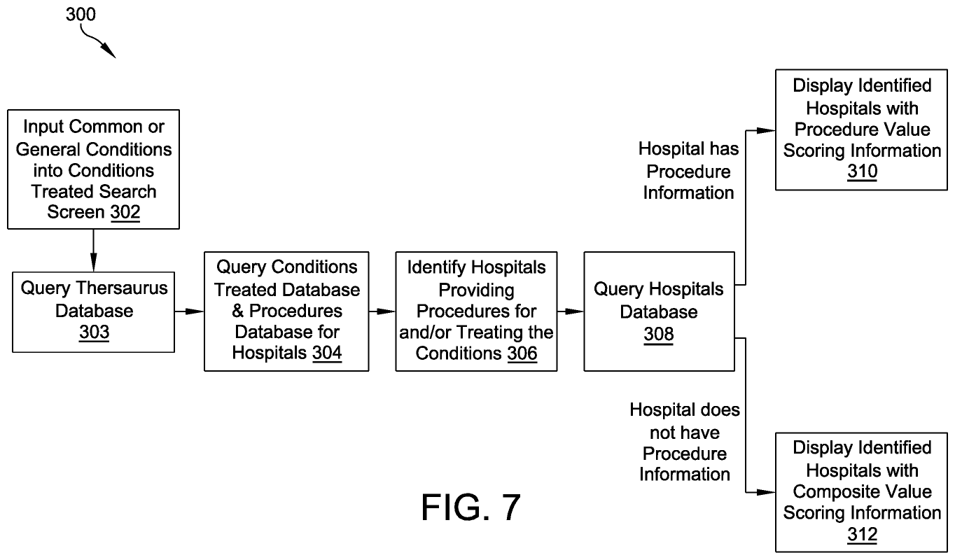

FIG. 7 provides a flowchart depicting an exemplary method (300) of searching for a hospital according to one or more conditions to be treated. In one embodiment, the method (300) may begin with the participant patient 116 inputting one or more common or general medical conditions into an interactive conditions treated search screen presented at the participant terminal 120 via the GUI 168 (302). The method (300) may continue with the search module 164 querying the thesaurus database 112₁₇ for the input conditions treated to determine if one or more normalized medical terms or search criteria apply for further searching (303). For example, the participant patient 116 may input the condition “high blood pressure,” and the search module 164 may query the thesaurus database 112₁₇ to determine the additional and/or replacement normalized condition “hypertension” for subsequent use in searching the procedures and/or conditions treated databases 112₈₋₉. Thus, the participant 116 may input a condition that is familiar to him or her, and the system will correlate that condition with one or more standardized or normalized medical terms for searching purposes, thus ensuring accurate searching regardless of the participant's familiarity with the technical medical vocabulary.

Next, the search module 164 may query the conditions treated database 112₉ and the procedures database 112₈ for hospitals associated with the treated conditions (304), applying both the input and the normalized conditions treated terms, to identify a list of one or more hospitals that provide one or more procedures associated with the input conditions or that treat the input conditions (306). The search engine may then query the hospitals database 112₁ (308) for procedure information associated with treating the input conditions for each of the identified hospitals (308). If the hospital(s) records include specific procedure information, the reporting module 166 may display the results to the participant 116 through a search results screen that includes specific procedure value scores via the GUI 168 at the participant terminal 120 (310). If the hospital(s) records do not include individual procedure information, results may be displayed to the participant through a search results screen that presents composite value score(s) for the hospital via the GUI 168 at the participant terminal 120 (312).

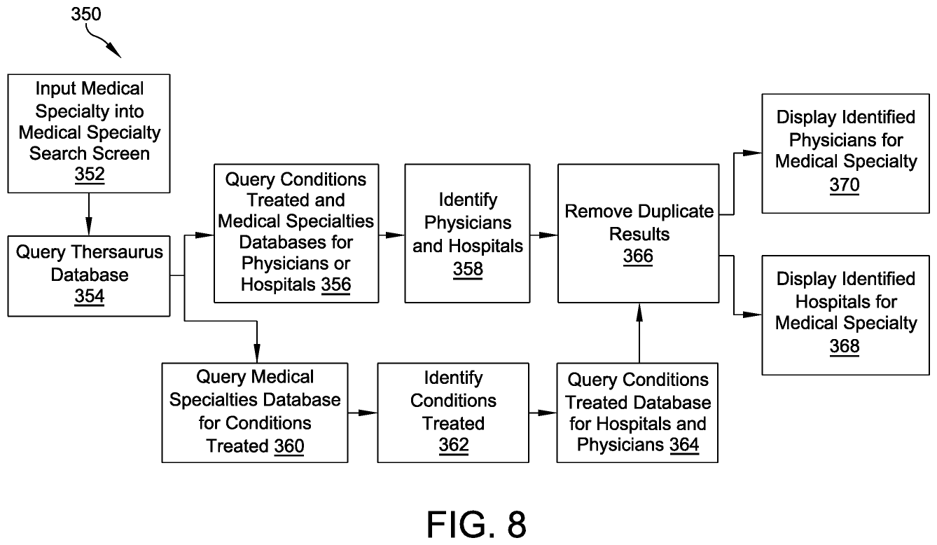

FIG. 8 provides a flowchart depicting an exemplary method (350) of searching for a physician or a hospital that practices one or more medical specialties. In one embodiment, the method (350) may begin with the participant patient 116 inputting, via the participant terminal 120, a medical specialty into a medical specialty search screen presented via the GUI 168 (352). The method (350) may continue with the search module 164 querying the thesaurus database 112₁₇ for the input medical specialty to determine if one or more normalized medical terms apply for further searching (354). For example, the participant patient 116 may input the medical specialty “pregnancy,” and the search module 164 may query the thesaurus database 112₁₇ to determine the additional and/or replacement normalized medical specialty “obstetrics” for subsequent use in searching the conditions treated and medical specialties databases 112₉₋₁₀.

After querying the thesaurus database 112₁₇ (354), the search module 164 may then simultaneously query the conditions treated database 112₉ and the medical specialties database for physicians and/or hospitals associated with the medical specialty (356), according to the input and the normalized terms, to identify a list of one or more physicians and/or hospitals that specialize in the input medical specialty (358). In parallel, the search module 164 may separately query the medical specialties database 112₁₀ for conditions treated that are associated with the medical specialty (360), according to the input and the normalized medical specialty terms, to identify a list of one or more conditions treated that are associated with the input medical specialty (362) before querying the conditions treated database 112₉ for a separate list of one or more hospitals and/or physicians that treat the identified conditions treated (364). Duplicates from the dual listings of hospitals and physicians may be removed (366), and the reporting module 166 may display the resulting listing of hospitals treating the input medical specialty to the participant 116 through a hospital search results screen via the GUI 168 at the participant terminal 120 (368). The resulting listing of physicians treating the input medical specialty may be displayed to the participant 116 through a physician search results screen via the GUI 168 at the participant terminal 120 (370).

Notably, the searching protocols discussed above may be tailored in any appropriate manner to suit the user searcher's needs. For example, searches may be further tailored or narrowed for geo location, filtered according to insurance providers accepted, filtered according a threshold value score for a procedure, medical specialty, condition treated, or provider, and so on.

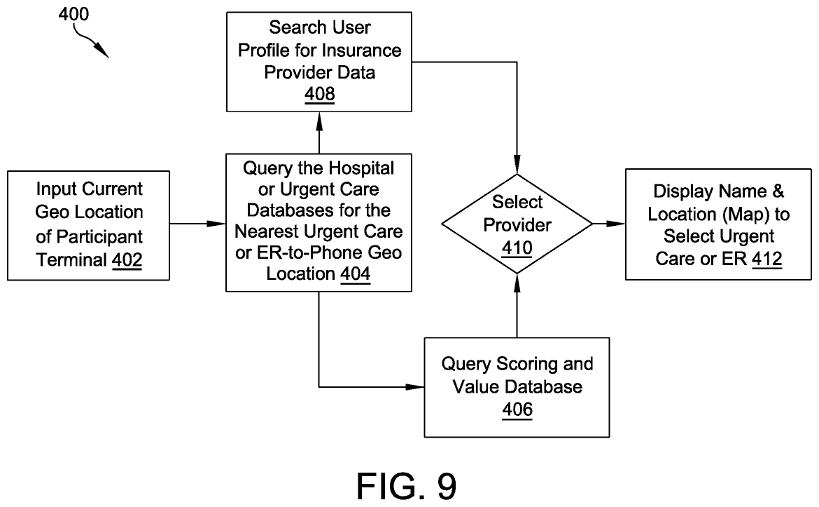

FIG. 9 provides a flowchart depicting an exemplary method (400) of searching for a hospital or urgent care facility having the nearest geo location relative to the participant terminal 120 or, in one embodiment, relative to a user-defined geo location. In one embodiment, the method (400) may begin with the participant patient 116 inputting his or her current geo location into a search hospitals screen or a search urgent care facilities screen presented at the participant patient terminal 120 via the GUI 168 (402). The geo location may be determined by a global positioning system (GPS) or other geo location technology running on the participant terminal 120 and in communication with the communication interface 154 of the software 150, or, alternatively, the location may be input manually in the form of an address or other geo location description such as a street corner, etc., into the hospital or urgent care search screen presented at the patient terminal 120 via the GUI module 160 and GUI 168.

Using the geo location of the terminal 120 (402), the search module 164 may query the hospital or the urgent care databases 112_{1, 3}, as appropriate, for a list of the hospital or urgent care facilities that are nearest to the geo location (404). After the geo location search (404), the search module 164 may query the scoring and value database 112₁₆ to determine the nearest hospital or urgent care facilities with the highest value scores (406), including composite scores or more granular scores. In parallel, the search module 164 may query the participant's profile within the participant database 112₁₃ to determine the nearest hospital or urgent care facilities that accept the participant patient's insurance carrier/plan (408). From the list of the nearest providers, the payer/insurance information, and the healthcare value data, the management engine may select the hospital or urgent care facility for the participant patient 116 that is nearest to the participant patient 116, that accepts the participant's insurance provider/plan, and that provides the highest relative value to the participant 116 (410). The result may be displayed to the participant 116 through a map results screen via the GUI 168 at the participant terminal 120 (412).

While the methods described above present several exemplary ways the search and value analysis tool may be used to improve outcomes and decrease costs, system components may be configured to enable a variety of use methodologies that provide the healthcare service provider 100, the participant patient 116, and the payer 118 with the feedback and information they need, individually or in combination, to compare providers, their procedure outcomes, and their costs in a manner that allows for improved decision-making for the patient, lower costs for the payer, and improved outcomes for all stakeholders. In a single tool, participants may navigate their medical needs, from searching for providers and healthcare facilities, to selecting the highest-value providers, to tracking medical histories, medications, providers, and more.

Prior to implementation of the present systems and methods, the technology of computerized healthcare services tracking and feedback, or existing computerized platforms for collecting, analyzing, tracking, searching, and reporting on the value of healthcare services was accomplished via silo-type systems that provided discrete information relevant only to a single payer or a single healthcare service provider or group of providers, or that collected and reported anecdotal reviews from patients. None of the existing technological solutions provided an integrated, regional or industry-wide technological solution that combines the collection of cost and quality data from healthcare service providers, with analysis of that data and reporting of that analysis in a comprehensive manner that creates a true feedback loop for payers and patients to use in selecting the best healthcare service provider for the patient and for healthcare service providers to use in improving the value of their services and keeping costs down.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

What is claimed is:

1. An integrated healthcare management tool for healthcare service providers, patients, and payers, the tool comprising:
a scoring and reporting server operating one or more scoring and reporting processors;
a database storing patient data, provider data, quality and cost data, and payer data; and
a management engine running on the scoring and reporting server, the management engine executing a database management module, a rule module including one or more scoring analysis algorithms, and a graphical user interface (GUI) module configured to display a GUI having a plurality of preconfigured, interactive screens to users at a provider terminal, a patient terminal, and a payer terminal, the management engine in communication with a healthcare service provider system of a healthcare service provider, the management engine:
receiving, from the healthcare service provider system, a set of quality and cost data associated with the healthcare service provider;
storing, in the database, the set of the quality and cost data; and
determining, based at least in part on the set of the quality and cost data, a value score for the healthcare service provider relative to a plurality of peer healthcare service providers.

2. The integrated healthcare management tool of claim 1, wherein the one or more of the scoring analysis algorithms comprise a plurality of predefined healthcare quality and cost measures.

3. The integrated healthcare management tool of claim 1, the management engine further:
displaying, via the plurality of the preconfigured, interactive screens of the GUI, the value score for the healthcare service provider at one or more of the provider terminal, the patient terminal, and the payer terminal.

4. The integrated healthcare management tool of claim 1, the management engine further:
receiving, from the patient terminal, a search request relating to a symptom, a healthcare procedure, a medical specialty, or a condition treated;
querying the database to process the search request; and
displaying, at the patient terminal via the plurality of the preconfigured, interactive screens of the GUI, a search result reflecting a highest value healthcare provider for the symptom, the healthcare procedure, the medical specialty, the condition treated, or a composite performance.

5. The integrated healthcare management tool of claim 4, wherein the highest value healthcare provider is associated with a highest value score for the symptom, the healthcare procedure, the medical specialty, the condition treated, or the composite performance.

6. The integrated healthcare management tool of claim 4, wherein:
the database comprises a synonym database storing a plurality of normalized medical terms; and
the querying the database to process the search request comprises querying the synonym database to equate the symptom, the healthcare procedure, the medical specialty, or the condition treated of the search request with one or more of the plurality of the normalized medical terms.

7. The integrated healthcare management tool of claim 1, the management engine further:
receiving, from the patient terminal, a patient geo location associated with the patient terminal and a search request relating to an emergency care facility having a provider geo location nearest to the patient geo location;
querying the database to process the search request; and
displaying, at the patient terminal via the plurality of the preconfigured, interactive screens of the GUI, a search result reflecting a highest value emergency care facility having the provider geo location nearest to the patient geo location.

8. The integrated healthcare management tool of claim 1, wherein the database is a HIPAA compliant database.

9. The integrated healthcare management tool of claim 1, wherein the set of the quality and cost data associated with the healthcare service provider comprises outcome information and cost information relating to a healthcare procedure, a medical specialty, a condition treated, or a composite performance of the healthcare service provider.

10. The integrated healthcare management tool of claim 1, wherein the payer is a patient, an employer, or a commercial or government health insurance provider.

11. A search and value analysis tool for healthcare providers, payers, and patients, comprising:
a scoring and reporting server including at least one scoring and reporting processor communicating over a network with a patient terminal, a healthcare service provider system, a provider terminal, a payer terminal, and a plurality of HIPAA compliant databases storing participant patient data, healthcare provider data, healthcare quality and cost data, payer data, and healthcare value data;
a value scoring module operating on the at least one scoring and reporting processor, the value scoring module executing one or more scoring analysis algorithms for determining a value score for a healthcare provider based on the healthcare provider data and the healthcare quality and cost data as compared to a plurality of predefined quality and cost measures; and
a graphical user interface (GUI) module operating on the at least one scoring and reporting processor, the GUI module displaying the value score for the healthcare provider upon a plurality of preconfigured screens presented at the patient terminal, the payer terminal, and the healthcare provider terminal.

12. The search and value analysis tool of claim 11, wherein the value score for the healthcare provider comprises a ranking of the healthcare provider in relation to a plurality of peer healthcare providers.

13. The search and value analysis tool of claim 11, wherein the value score for the healthcare provider relates to a medical procedure, a medical specialty, a condition treated, or a composite performance of the healthcare provider.

14. The search and value analysis tool of claim 11, further comprising:
a search module operating on the at least one scoring and reporting processor, the search module executing one or more search algorithms to query the plurality of the HIPAA compliant databases in response to a search request including one or more search criteria received from the patient terminal; and
a reporting module operating on the at least one scoring and reporting processor, the reporting module executing one or more reporting algorithms to display a search result via the plurality of the preconfigured screens presented at the patient terminal via the GUI module, the search result comprising a highest value medical provider that fulfills the one or more of the search criteria.

15. The search and value analysis tool of claim 14, wherein the one or more of the search criteria relate to one or more of a physician, a healthcare facility, a medical procedure, a medical specialty, a medical symptom, and a geo location.

16. The search and value analysis tool of claim 14, the search module further executing the one or more search algorithms to match the one or more of the search criteria of the search request with one or more normalized medical search criteria.

17. A method of technically evaluating a healthcare provider as compared to a plurality of peer healthcare providers using a search and value analysis tool having a scoring and reporting processor in communication with a database storing healthcare provider data and quality and cost data, the scoring and reporting processor including a management engine executing a value scoring module including one or more scoring analysis algorithms and a graphical user interface module (GUI) module configured to display a GUI having a plurality of preconfigured screens at a provider terminal and at a patient terminal, the method comprising:
receiving, at the scoring and reporting processor from a healthcare service provider system, a set of quality and cost data relating to the healthcare provider;
determining, via the value scoring module, a value score for the healthcare provider based on the set of the quality and cost data as compared to a plurality of predefined quality and cost measures; and
displaying, via the plurality of the preconfigured screens of the GUI module, the value score at one of the provider terminal and the patient terminal.

18. The method of claim 17, wherein the value score comprises a composite value score, a procedure value score, a medical specialty value score, or a condition treated value score for the healthcare provider.

19. The method of claim 17, the management engine further executing a search module including one or more search algorithms, the method further comprising:
receiving, via one of the plurality of the preconfigured screens displayed at the patient terminal, a search request including one or more input search criteria relating to one or more of a healthcare provider, a symptom, a medical specialty, a condition treated, and a geo location;
determining, via the search module, one or more normalized search criteria that equate to the one or more input search criteria; and
determining, via the search module, a search result comprising a highest value medical provider that fulfills the one or more of the input search criteria and the one or more of the normalized search criteria.

20. The method of claim 19, further comprising displaying, via the plurality of the preconfigured screens of the GUI module, the search result at the patient terminal.
